(19) United States
(12) Patent Application Publication
(10) Pub. No.: US 2008/0243110 A1
(43) Pub. Date: Oct. 2, 2008

(54) Laser Apparatus for Medical Treatment of Skin Disease

(76) Inventors: Uk Kang, Gunpo-si (KR); Guang Hoon Kim, Suyeong-gu (KR); Dae Sic Lee, Ansan-si (KR); Geun Hie Rim, Changwon-si (KR)

Correspondence Address: FROMMER LAWRENCE & HAUG, 745 FIFTH AVENUE- 10TH FL., NEW YORK, NY, 10151, US

(21) Appl. No.: 12/058,828

(22) Filed: Mar. 31, 2008

(30) Foreign Application Priority Data
Mar. 31, 2007 (KR) 10-2007-0032033

(51) Int. Cl. A61B 18/20

(52) U.S. Cl. 606/9

(57) ABSTRACT

The present invention provides a laser apparatus for medical treatment of skin disease. The laser apparatus comprising: a laser generator 12 for generating a laser beam of a predetermined wavelength range; a handpiece 14 having a microlens array 11 installed therein for forming a plurality of laser spots; and an optical energy transfer element 13 connected to the laser generator and the handpiece for transferring optical energy to a to-be-treated object.

### CROSS-REFERENCE TO RELATED APPLICATION

This application claims the benefit under 35 U.S.C. §119 (a) of Korean Patent Application No. 10-2007-0032033 filed on Mar. 31, 2007, the entire contents of which are incorporated herein by reference.

### BACKGROUND OF THE INVENTION

(a) Field of the Invention

The present invention relates to a laser apparatus for medical treatment of skin disease. More particularly, the present invention relates to a laser apparatus for medical treatment of skin disease, which improves a general treatment method using a laser in which energy is transferred from a scar or skin fold region up to a given depth of the skin when the skin is damaged to thereby lead to ineffectiveness in the treatment of the damaged skin, so that much more heat energy is transferred to a to-be-treated object using a microlens array consisting of multiple microlenses having different focal lengths so as to allow much more energy to be transferred to a dermis layer to thereby relatively effectively treat the ski disease as compared to an existing treatment method.

(b) Background Art

In general, a treatment using a laser is widely used for the treatment of skin disease such as acne, scar, skin fold and the like.

And, a skin treatment method using a new laser is currently spotlighted which several hundreds to hundred thousands of micro treatment zones are formed per 1 cm² and medical treatment is given to each of the micro treatment zones using a laser.

A laser beam from a laser is irradiated onto a plurality of laser spots formed on a to-be-treated object so as to irradiate the laser beam onto the several hundreds to hundred thousands of micro treatment zones. Such laser spots are formed using a conventional laser scanner.

As shown in FIG. 6, a skin treatment method using the laser is performed in such a fashion that micro treatment zones are formed and energy is transferred to a given depth of the micro treatment zones through a microlens array 11 to thereby treat the damaged skin.

However, such a conventional skin treatment method using the laser entails a problem in that energy is transferred to a given depth of the micro treatment zones irrespective of the state of the skin, i.e., a to-be-treated object upon the formation of the micro treatment zones, at which time the energy is excessively transferred due to the overlapping of heat energy.

The information disclosed in this Background of the Invention section is only for enhancement of understanding of the background of the invention and should not be taken as an acknowledgment or any form of suggestion that this information forms the prior art that is already known to a person skilled in that art.

### SUMMARY OF THE INVENTION

The present invention has been made in an effort to solve the above problems occurring in the prior art, and it is an object of the present invention to provide a laser apparatus for medical treatment of skin disease, in which when the skin is damaged, energy is transferred to various depths of the skin using a microlens array consisting of multiple microlenses having different focal lengths and simultaneously a plurality laser spots are implemented, in which heat is transferred to the inside of dermis using the laser spots to thereby minimize a heat damage to epithelium and epidermis, and in which when optical energy is transferred to the dermis from the microlens array consisting of multiple microlenses having different focal lengths so as to be converted into heat energy at the inside of the dermis without being overlapped.

In order to accomplish the above object, according to a preferred embodiment of the present invention, there is provided a laser apparatus for medical treatment of skin disease, comprising: a laser generator for generating a laser beam of a predetermined wavelength range; a handpiece having a microlens array installed therein for forming a plurality of laser spots; and an optical energy transfer element connected to the laser generator and the handpiece for transferring optical energy to a to-be-treated object.

In a preferred embodiment, the microlens array comprises a plurality of microlenses which are arranged regularly and have different focal lengths.

In a preferred embodiment, the microlenses have different focal lengths by changing a radius of curvature of each of the microlenses.

Also, the microlenses have different focal lengths by changing a pitch of each of the microlenses.

Further, the handpiece comprises a diaphragm mounted therein so as to be disposed in parallel with the microlens array, so that the amount of the optical energy incident to the microlens array is adjusted by means of the diaphragm.

### BRIEF DESCRIPTION OF THE DRAWINGS

FIG. 1 is a diagrammatic view showing a laser apparatus for medical treatment of skin disease according to one embodiment of the present invention;

FIG. 2 is a schematic showing an array of microlenses having different focal lengths according to one embodiment of the present invention;

FIG. 3 is a top plan view showing a diaphragm for adjusting the region of a pin hole according to one embodiment of the present invention;

FIG. 4 is a graph showing an absorption coefficient and a scattering coefficient of a dermis layer of the skin at a wavelength which is the most suitable for producing collagen;

FIG. 5 is a graph showing water absorption according to a variation of wavelength;

FIGS. 6a and 6b are cross-sectional views showing a microlens array consisting of a plurality of microlenses having different pitches and radiuses of curvature according to a preferred embodiment of the present invention;

FIG. 7 is a cross-sectional view for explanting a sum ‘h’ of a lens sag and a substrate thickness;

FIG. 8 is a view showing a radius of a laser beam at a lens focus; and

FIG. 9 is a schematic view showing a conventional medical treatment method and a medical treatment method of the present invention using a laser.

Reference numerals set forth in the Drawings includes reference to the following elements as further discussed below:

| | |
|---|---|
| 10: microlens | 11: microlens array |
| 12: laser generator | 13: optical energy transfer element |
| 14: handpiece | 15: epidermis |
| 16: dermis | 17: diaphragm |
| 18: subcutaneous fat | 19: scar or skin fold |

### DETAILED DESCRIPTION

Reference will now be made in detail to the preferred embodiment of the present invention, examples of which are illustrated in the drawings attached hereinafter, wherein like reference numerals refer to like elements throughout. The embodiments are described below so as to explain the present invention by referring to the figures.

Now, a preferred embodiment of according to the present invention will be described hereinafter in detail with reference to the accompanying drawings.

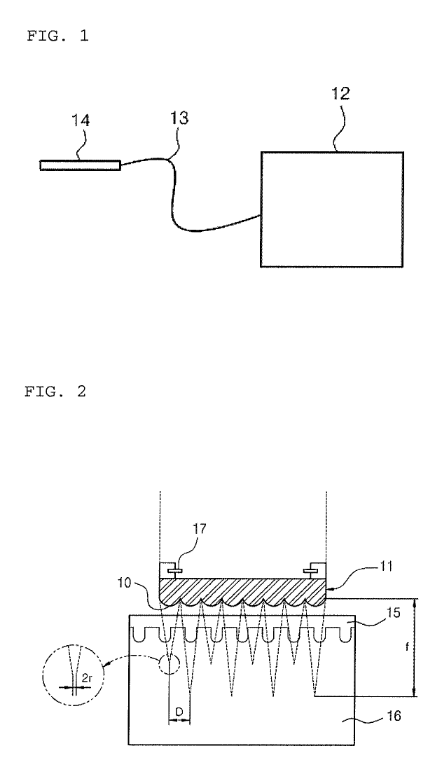

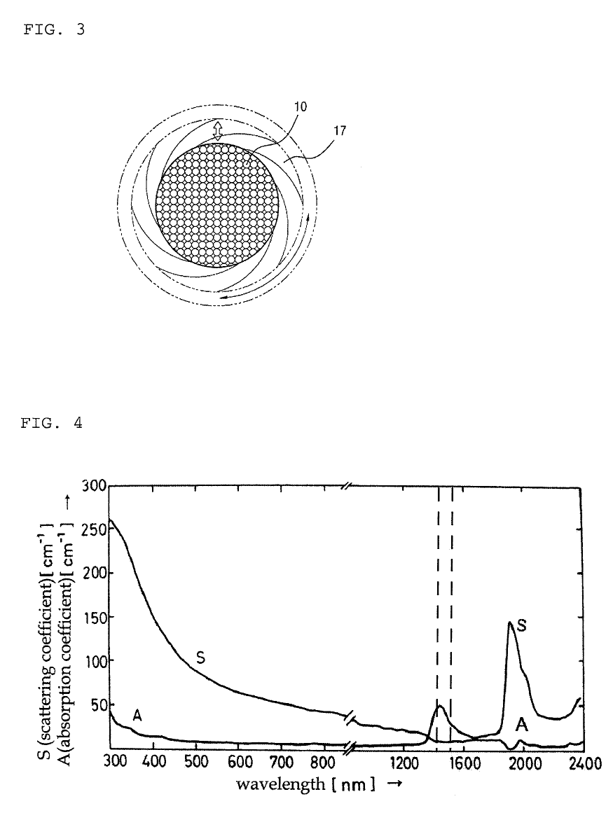

FIG. 1 is a diagrammatic view showing a laser apparatus for medical treatment of skin disease according to one embodiment of the present invention, FIG. 2 is a schematic showing an array of microlenses having different focal lengths according to one embodiment of the present invention, and FIG. 3 is a top plan view showing a diaphragm for adjusting the size region of a pin hole according to one embodiment of the present invention.

The present invention features that multiple laser spots are formed to have different depths on a to-be-treated object by a microlens array 11 consisting of a plurality of microlens having different focal lengths 10.

The present invention includes a laser generator 12 for generating a laser beam of a predetermined wavelength range so as to treat the damaged skin such as scar or skin fold 19, an optical energy transfer element 13 connected to the laser generator 12 for transferring optical energy to a to-be-treated object, and a handpiece 14 having a microlens array 11 consisting of a plurality of microlenses having various focal lengths installed therein so as to form a plurality of laser spots.

The laser generator 12 generates a single laser beam of a predetermined wavelength range for the treatment of the skin. The optical energy transfer element 13 serves to interconnect the handpiece 14 and the laser generator 12 for guiding the laser beam generated from the laser generator 12 to the microles array 11 disposed inside the handpiece 14.

The main tissue inside the skin is formed of a gelatinous tissue of a skin layer, and the ratio of collagen (protein=fiber) occupying the inside of the skin is approximately 90%. A colloidal element is fully filled between fibers.

As one grows older, the activity of the skin tissue is gradually degenerated and the amount of collagen is reduced and hardened. Also, the moisture retention capacity of the skin tissue is degraded and the skin loses elasticity. The skin tissue is even ruptured.

As a result, the skin is susceptible to irritation and damage, and becomes dry so that black speckles and fine wrinkles are caused on the surface thereof.

Collagen is a protein which is contained in a large amount in each region of the human body and serves as a ring that interconnects cells. Collagen also serves to maintain glassy and elasticity of the skin.

Collagen occupies one third of the total protein constituting the human body and plays an important role in promotion of blood circulation of our whole body.

Collagen interconnects a cell and a cell and nutrients absorbed in the small intestine are transferred to each cell by means of collagen. In addition, a metabolic waste product is carried to a bloody vessel through collagen so as to be discharged to the outside of the human body.

The skin comprises an epidermis 15 and a dermis 16 under keratin.

The epidermis 15 consists of four layers positioned under a sebaceous film, and a skin cell is produced in a base layer which is one of the four layers.

The produced skin cell is moved toward an upper layer while changing its shape and then is gradually changed into a keratin. Ultimately, the keratin becomes the dirt which will in turn be removed from the skin. Likewise, the skin is continuously produced and changed.

The dermis 16 supports the base layer playing an important role in producing the skin cell. The dermis 16 is a key place which influences the moisture retention power of the epidermis 15, and maintains elasticity of the skin to thereby make the skin taut.

A large portion of the dermis 16 is occupied by collagen. The reason why the skin is aged, or chloasma, wrinkle, freckle and the like occur in a large amount on the skin is because collagen is insufficient or its quality is degraded.

In the meantime, the correlation between the skin and the optical energy is directly related to a wavelength of the optical energy as well as skin absorption and scattering of the optical energy.

Thus, the production of collagen of the dermis 16 must be first performed in order to medically treat scar and pigmentation of skin.

The wavelength the most suitable for the production of collagen is within a range between 1400 nm and 1500 nm as shown in FIG. 4. The reason for this is that an absorption coefficient (A) of the dermis layer is the highest and a scattering coefficient (B) of the dermis layer is the lowest in the above wavelength range.

Also, in case where the optical energy absorbed into the skin is converted into heat energy, if the optical energy is not absorbed into moisture inside the skin, thermal damage is applied to the skin tissue surrounding the skin surface, leading to a reduction in a medical treatment effect.

As shown in FIG. 5, since a high water absorption is exhibited in the wavelength ranging from 1400 nm from 1500 nm, heat can be transferred to only a to-be-treated region. The use of the wavelength of the above range enables the selective treatment of the skin.

The microlens array 11 is a structure in which a plurality of microlenses 10 is arranged regularly. The microlens array 11 is installed in the handpiece 14 so as to be oriented perpendicular to the progress direction of a laser beam. The microlenses 10 are convex lenses and irradiate the optical energy introduced to the handpiece 14 from the laser generator 12 through the optical energy transfer element 13 onto a to-be-treated object while having different focal lengths.

In this case, the focal lengths (f) of the respective microlenses may be made different from each other by changing pitches (D) and radiuses (R) of curvature of the microlenses 10.

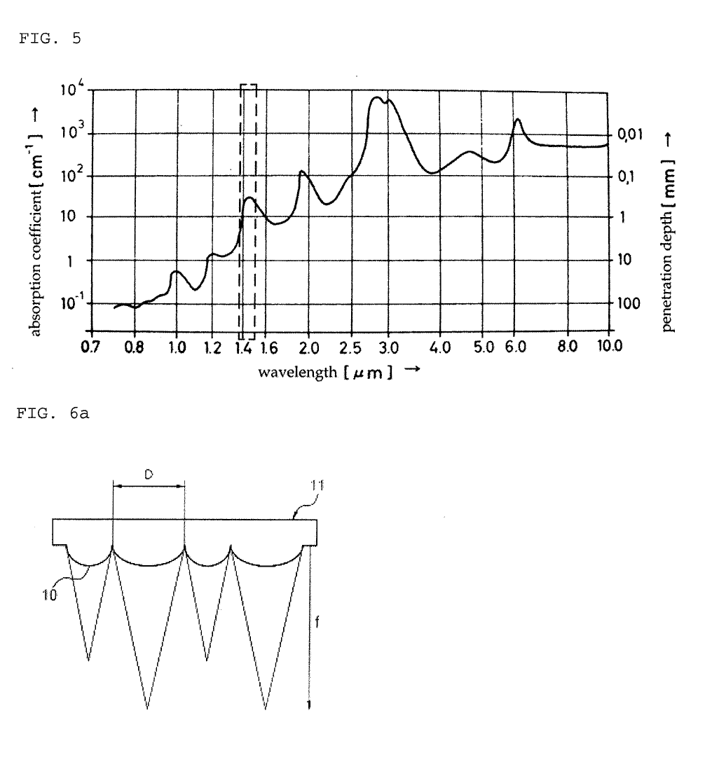

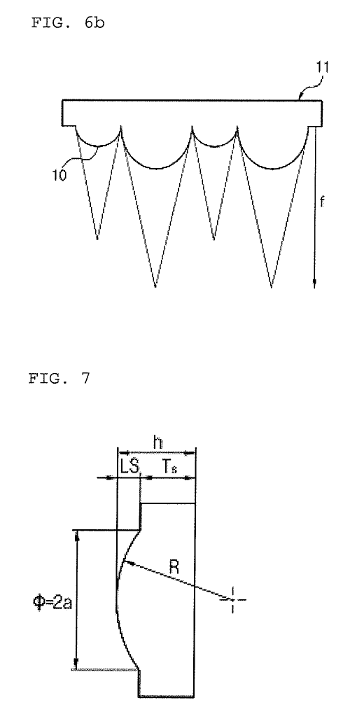

FIG. 6a shows a cross-sectional view of a microlens array consisting of a plurality of microlenses having different pitches according to a preferred embodiment of the present invention, and FIG. 6b shows a cross-sectional view of a microlens array consisting of a plurality of microlenses having different radiuses of curvature according to a preferred embodiment of the present invention.

As shown in FIGS. 6a and 6b, when the radius (R) of curvature is changed, the pitches (D) of the respective microlenses may be identical or different. Basically, the focal length (f) of the microlens varies depending on a change of the radius (R) of curvature of the microlens, which is expressed by the following Equation 1:

$$f = \frac{R}{n - 1} , R = \frac{{a}^{2}}{2 h} \frac{+ ( k + 1 ) h}{2} , \qquad [ Equation 1 ]$$

where f is a focal length, R is a radius of curvature and n is a refraction index. Also, his a sum of a lens sag and a substrate thickness, a is a radius of a lens and k is a conical constant which is defined as follows:

$$k = \{ \begin{matrix}0 \text{:} & SphericalLens \\ 1 \text{:} & ParabolicLens \\ - {( n )}^{2} \text{:} & HyperbolicLens\end{matrix} \\ r = \frac{λ f}{π a} \qquad [ Equation 2 ]$$

where r is a radius of a laser beam at a focus, λ is a wavelength of a laser beam, f is a focal length of a lens and a is a radius of the lens.

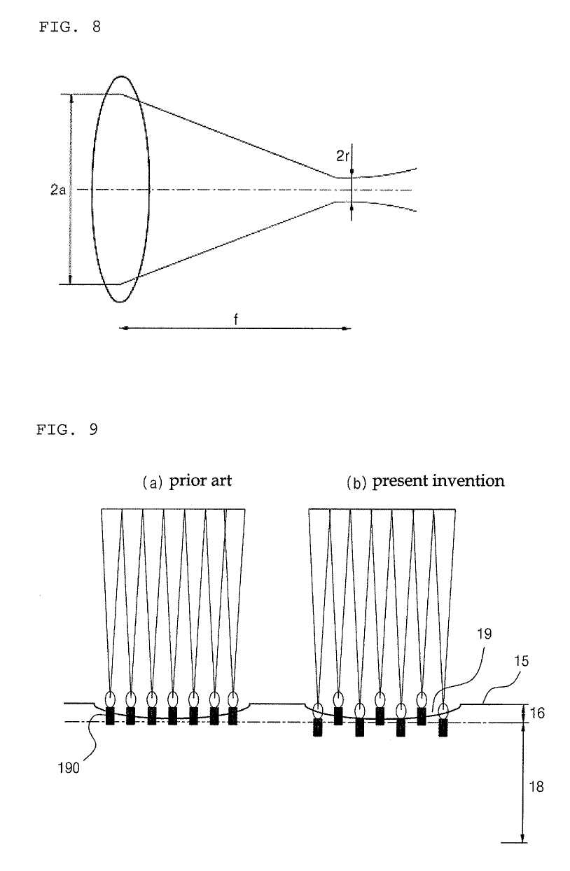

FIG. 7 is a cross-sectional view for explanting a sum ‘h’ of a lens sag and a substrate thickness, FIG. 8 is a view showing a radius ‘r’ of a laser beam at a lens focus, and FIG. 9 is a schematic view showing a conventional medical treatment method and a medical treatment method of the present invention using a laser. In FIG. 9, non-explained reference numeral 190 denotes a to-be-treated region.

The microlens array 11 comprising a plurality of microlenses having different focal lengths can be used to change the depth of laser beam penetrated into the dermis 16 depending on the depth of a to-be-treated lesion of a patient so as to treat the lesion.

As shown in FIG. 3, the use of a diaphragm 17 can adjust an area of the microlens array to which the laser beam is incident.

The diaphragm 17 is mounted above the microlens array 11 so that it can be rotated to regulate the amount of the laser beam incident to the microlens array 11. In this case, the diaphragm 17 is a device which adjusts the diameter of the laser beam incident to the microlens array to regulate the amount of the optical energy of the laser beam. When the diaphragm is opened completely, a fine treatment area is formed as much as the size of the microlens array and the number of microlenses. On the other hand, when the diaphragm is closed, the optical energy is transferred to the microlens array as much as the size of the closed diaphragm such that the fine treatment area is decreased as much as the size of the closed diaphragm. Thus, the size of the to-be-treated region can be adjusted through the operation of the diaphragm.

Also, the microlens 10 shown in FIG. 3 has a circular shape but may be formed in a quadrangular shape.

The operation of the laser apparatus for medical treatment of skin disease according to the present invention as constructed above will be described hereinafter.

A single laser beam outputted from the laser generator 12 is incident to the microlens array 12 by the guidance of the optical energy transfer element 13.

The laser beam incident to the microlens array 12 is split into a plurality of lights by the plurality of microlenses 10, leading to simultaneous generation of a plurality of laser spots.

Here, pin holes are formed in the dermis 16 using the laser spots so as to transfer heat to the inside of the dermis 14, thereby minimizing heat damage to the epithelium and the epidermis 15.

In addition, the pin holes formed in the dermis 16 according the present invention have different depths so as to transfer heat to the inside of the dermis. The transferred heat allows energy to be applied to the dermis 16 without being overlapped, thereby improving heat efficiency in the medical treatment of skin disease. Moreover, the inventive laser apparatus employs the microlens array 11 including a plurality of microlenses having different focal lengths (f) so as to allow much more heat energy to be transferred to the dermis 16 of a to-be-treated object as compared to the conventional treatment method in which energy is transferred to a uniform depth of the dermis at a scar or a skin fold 19 when the skin is damaged.

Furthermore, the inventive laser apparatus has an advantage in that the microlens array 11 including a plurality of microlenses having different focal lengths is configured as a single system to thereby reduce a price of a product and is easy to remove and mount.

As described above, according to a laser apparatus for medical treatment of skin disease according to the present invention, multi laser spots are formed in a to-be-treated object using a microlens array consisting of multiple microlenses having different focal lengths, and the optical energy is transferred to different depths of the to-be-treated object to which the laser beam is penetrated using the laser spots, thereby improving heat efficiency in the medical treatment of skin disease.

The invention has been described in detain with reference to preferred embodiments thereof. However, it will be appreciated by those skilled in the art that changes may be made in these embodiments without departing from the principles and spirit of the invention, the scope of which is defined in the appended claims and their equivalents.

1. A laser apparatus for medical treatment of skin disease, the laser apparatus comprising:
a laser generator for generating a laser beam of a predetermined wavelength range;
a handpiece having a microlens array installed therein for forming a plurality of laser spots; and
an optical energy transfer element connected to the laser generator and the handpiece for transferring optical energy to a to-be-treated object.

2. The laser apparatus of claim 1, wherein the microlens array comprises a plurality of microlenses which are arranged regularly and have different focal lengths.

3. The laser apparatus of claim 1, wherein the microlenses have different focal lengths by changing a radius of curvature of each of the microlenses.

4. The laser apparatus of claim 1, wherein the microlenses have different focal lengths by changing a pitch of each of the microlenses.

5. The laser apparatus of claim 1, wherein the handpiece comprises a diaphragm mounted therein so as to be disposed in parallel with the microlens array, so that the amount of the optical energy incident to the microlens array is adjusted by means of the diaphragm.

6. The laser apparatus of claim 2, wherein the microlenses have different focal lengths by changing a radius of curvature of each of the microlenses.

7. The laser apparatus of claim 2, wherein the microlenses have different focal lengths by changing a pitch of each of the microlenses.

8. The laser apparatus of claim 2, wherein the handpiece comprises a diaphragm mounted therein so as to be disposed in parallel with the microlens array, so that the amount of the optical energy incident to the microlens array is adjusted by means of the diaphragm.
